(19) United States
(12) Patent Application Publication
(10) Pub. No.: US 2012/0088254 A1
(43) Pub. Date: Apr. 12, 2012

(54) WHOLE BLOOD ASSAY OF AN INTRACELLULAR BIOMARKER OF A CELL SIGNALLING PATHWAY USE IN MEASURING THE ACTIVATION OF A PREDETERMINED CELL POPULATION

(75) Inventors: Maxime Moulard, Aubagne (FR); Daniele Boulay Moine, Saint Victoret (FR)

(73) Assignee: BIOCYTEX, Marseille (FR)

(21) Appl. No.: 13/377,592

(22) PCT Filed: Jun. 10, 2010
(86) PCT No.: PCT/FR10/51164

§ 371 Date: Dec. 12, 2011

(30) Foreign Application Priority Data
Jun. 12, 2009 (FR) 0902885

(51) Int. Cl. G01N 33/573; G01N 33/53

(52) U.S. Cl. 435/7.25; 435/7.92

(57) ABSTRACT

A method for assaying by ELISA, carried out on a whole blood sample, of the cellular activation state of a predetermined population of cells of the sample, includes determining the phosphorylation state of an intracellular protein involved in a cell signaling pathway (biomarker). A particular application of the method for assaying the phosphorylation state of an intracellular protein involves monitoring agonist agents or antagonist agents of cellular activation of cellular populations present in a whole blood sample. Such an application may in particular contribute to therapeutic monitoring of patients, or it may be useful in drug screening. In particular, the application pertains to the context of the exploration of hemostasis, in particular blood coagulation, from an observation of the activation of platelets contained in whole blood samples.

The invention relates to a method for in vitro assay of an intracellular biomarker involved in a cell signalling pathway, carried out on a whole blood sample. The invention also relates to the use of said method in measuring the activation of cells of a predetermined cell population contained in the blood sample.

The application relates to the exploration of physiological processes that may occur upon activation of cells present in the blood. Such cells may be cells naturally present in the blood or cells found in the blood circulation as a consequence of a particular physiological state, in particular a pathological state.

It has been observed that cell activation is accompanied by a modification to the state of certain intracellular proteins, in particular a modification of the phosphorylation state of proteins belonging to a cell signaling pathway. The invention relates to the analysis of such proteins and of their phosphorylation state, to act as biomarkers for the activation of a cell population.

Thus, the application is aimed at an assay method in which the intracellular protein involved in a cell signaling pathway, in particular in signal transduction, is a protein the expression of which is regulated by phosphorylation. The protein the phosphorylation state of which is observed is thus a biomarker for cell activation and as a consequence for cell activity.

A particular application of the method for assaying the phosphorylation state of an intracellular protein in accordance with the invention concerns monitoring agonistic agents or antagonistic agents for the cellular activation of cell populations present in a whole blood sample. Such an application may in particular contribute to therapeutic monitoring of patients, or it may be useful for drug screening.

In particular, the context of the application is the exploration of hemostasis and in particular blood coagulation, from observing the activation of cell populations contained in whole blood samples. Cell activation is in fact a process that occurs when reactions leading to blood coagulation are triggered. In particular, the activation of blood platelets observed following their in vivo adhesion to sub-endothelial structures or the action of an external in vitro stimulus (ADP, thrombin, collagen, etc) may lead to their aggregation and under these conditions, allow the reactions of the coagulation cascade to occur properly, allowing the formation of a thrombus due to stabilization of the platelet aggregate by means of the fibril network.

Thus, the invention is more particularly applicable to the analysis of the phosphorylation state of an intracellular protein expressed in the platelets (thrombocytes) contained in a sample of whole blood, in order to determine the activation state of said platelets and more generally to contribute to exploration of platelet activity.

The invention is of particular application in the medical field linked to diseases of the vascular system, in particular for the investigation of active substances, or monitoring substances that are effective in the prevention or treatment of diseases of the vascular system. A particular application of the method of the invention thus consists of monitoring the response of platelets to known or potential platelet anti-aggregation agents. Thus, the method of the invention can, for example, be used to screen biological or other resistances to platelet anti-aggregating agents, to adapt posologies for platelet anti-aggregating agents administered to a patient, or to contribute to determining whether a therapeutic treatment should be interrupted or continued.

In the context of assaying the phosphorylation state of an intracellular protein expressed in platelets, particular examples of pathologies that might benefit from the method of the invention with regard to their diagnosis or monitoring, including for the identification of compounds for their treatment or control are acute coronary syndromes as a consequence of coronary atherosclerosis, such as myocardial infarction, coronary thrombosis, coronary occlusion, cerebral vascular accidents of ischemic origin, type II diabetes, drepanocytosis, etc.

Platelet activation is a consequence of triggering the thrombotic process, for example following a vascular lesion or rupture of an arterial plaque. The activated platelets adhere to the vascular wall then aggregate reversibly then irreversibly, leading to physiological repair of the lesion in the vascular endothelium or, in a pathological situation, to obstruction of the vessel.

Among the molecular partners identified in the platelet activation process and of interest in the context of implementation of the invention, the role of the nucleotide ADP (adenosine diphosphate), released by the dense granules of the platelets, after having been secreted in damaged cells, or in response to coagulation factors such as thrombin, has been observed. Stimulation of platelets by ADP leads to the formation of unstable platelet aggregates. One of the platelet ADP receptors is a purinergic type P2 receptor designated P2Y12 (Hollopeter G et al, Nature 2001: 409: 202-7), which is responsible for the selective activation of certain pathways (cascades) for intracell signal transduction. The P2Y12 receptor is responsible for the stability of the platelet aggregates formed.

Platelet activation may be stimulated by physiological agonists such as prostaglandins (PGE), in particular the prostaglandin PGE1 or PGE2, by PGI prostaglandins such as PGI2 or PGDs such as PGD2. Platelet activation by PGE1 activates phosphorylation of the protein VASP (vasodilator-stimulated phosphoprotein). The phosphorylation of VASP occurs at serine 239 (in the sequence published by the NCBI with accession number NP003361.1 in the version dated May 10 2009), in an amount proportional to the intracellular concentration of cAMP (cyclic adenosine monophosphate) when activation is initiated by PGE1, for example, and/or cGMP (cyclic guanosine monophosphate) when activation is initiated by NO (nitric oxide)-donating compounds, for example.

The intracellular concentrations of cAMP or cGMP are regulated by the concerted action of adenylyl cyclase and guanylyl cyclase respectively and of phosphodiesterases. Kinases are key enzymes in the regulation of cellular activation. Said kinases are numerous and responsible for the phosphorylation of membrane or intracellular proteins by transfer of the phosphate groups of ATP to the serine, threonine or thyrosine residues of the protein substrates. This post-translational modification regulates a large number of activities of the cell cycle and abnormal phosphorylation may be involved in many pathological states. Certain kinases are the direct or indirect targets of biologically active molecules, in particular active substances forming part of the composition of drugs, or candidates for the definition of therapeutic active principles or molecules of diagnostic interest. These include the kinases AKT (sometimes termed PKB), ERK, JAK2, BCR-ABL (principally present in certain leukemia tumor cells), PKA, PKG. As a consequence, in the context of the invention, the kinases or intracellular proteins forming the substrates of said kinases may be revealed to be biomarkers of interest in following the change in activity of the kinases.

The VASP protein of platelets is a substrate for certain of said kinases, in particular PKA (cAMP-dependent protein kinase) and PKG (cGMP-dependent protein kinase). VASP plays a pivotal role in the regulation of the dynamics of the platelet cytoskeleton and is considered in this precise case to be a biomarker for the activity of PKA and/or PGK, but also for the whole of the cell signaling cascade occurring upstream.

Further, phosphorylation of the VASP protein is also controlled by another molecule in circulating blood, the nucleotide ADP. When ADP interacts with the platelet receptor P2Y12, this latter is occupied and as a consequence, phosphorylation of the VASP protein (vasodilator-stimulated phosphoprotein) induced by the physiological agonists is inhibited or limited.

Inhibition or limitation/modification of VASP phosphorylation includes dephosphorylation, total or partial, of the VASP protein previously induced by an agonist for phosphorylation, or total or partial prevention of said phosphorylation.

The protein AKT is a kinase the enzymatic expression of which is directly dependent on the phosphorylation state of the active site of the amino acid Ser 473 (serine in position 473 in the sequence identified in the NCBI database with accession number NP001014431.1 in the version dated May 22, 2009). AKT is also considered to be a cellular mediator or a cell signaling protein.

In the platelet, the activation by thrombin of the receptor PAR-1 induces phosphorylation of AKT. Similarly, activation by ADP of the receptor P2Y12 also appears to induce the phosphorylation of the amino acid Ser 473. As a consequence, the phosphorylation state of serine 473 is considered to be a biomarker of the state of the thrombin receptor (par1) or the ADP receptor, P2Y12.

Thus, it is important to measure the activation state of blood cell populations and in particular of platelets in the blood of patients presenting a risk of vascular pathology, or of patients suffering from such pathologies, and also of patients treated for such pathologies, in particular to evaluate the interference of administered active substances on the platelet activation process. It is also important to measure the activation state of platelets in samples from whole blood in order to test candidate substances for the design of agonist or antagonists for the activation of platelets, in particular by specific interaction with platelet receptors.

To this end, the invention proposes means that are based on observing the phosphorylation of intracellular proteins expressed in the platelets.

Means have already been described in the prior art to analyze platelet activation by determining the modifications to the phosphorylation state of intracellular proteins expressed in platelets. The kit designated PLT VASP/P2Y12 from Biocytex (Marseille, France), for example, proposes an in vitro test carried out on samples of whole blood and consisting of determining the activation state of platelets by measuring the phosphorylation state of the intracellular protein VASP (vasodilator-stimulated phosphoprotein). In the context of that test, phosphorylation of the protein VASP is measured using dual-color flow cytometry in order to compare the two test conditions (a portion of the sample undergoes activation of VASP using prostaglandin PGE1 and another portion undergoes activation using PGE1 and ADP). The analysis is made by detecting fluorescence and calculating a platelet reactivity index. Flow cytometric analysis requires isolating, on the cytogram, the cellular cloud including the platelets in order to prevent other cell groups of the blood or cell debris from interfering with the measurement. Determining the conditions for accomplishing cytometric analysis and carrying out that analysis may, however, represent a source of difficulty.

The invention proposes an alternative to carrying out a cytometric analysis, in whole blood, of the phosphorylation state of intracellular proteins, in particular the platelet VASP protein. In accordance with this alternative, the analysis is carried out using an ELISA type immunoenzymatic test.

To this end, the inventors have determined the conditions for activation and also for inhibition of platelets in whole blood, which means that a specific measurement of platelet activation mediated by a predetermined platelet receptor can be carried out.

In the context of the invention, therefore, the inventors have defined a test that is distinguished from biochemical and cellular principles wherein the signal in an ELISA analysis is always measured following activation of a receptor from a homogeneous cell population that has been separated from the other components and cells of the sample, or which has been verified as the only target of the analysis (for example a cultured tumor cell line or purified lymphocytes or lymphocytes isolated from blood).

By dispensing with this necessity, the inventors have thus demonstrated that the reasons restricting the use of tests for monitoring the platelet activity in a preparation of a PRP (platelet-rich plasma) may be overcome.

The present invention concerns a method for assaying, by ELISA carried out on a sample of whole blood, the cellular activation state of a predetermined population of cells of the sample, comprising determining the phosphorylation state of an intracellular protein involved in the cell signaling cascade (biomarker).

Thus, the invention proposes means for determining, in an ELISA analysis, the phosphorylation state of intracellular proteins expressed in a predetermined cell population in a sample of whole blood, in particular in platelets. One of the intracellular proteins thus used as a marker for the activation state of the platelets is the protein VASP. Another of said proteins is the protein AKT.

In the context of the present application, the term “sample of whole blood” is applied to a sample of blood taken from a human or animal subject, which sample is not fractionated prior to carrying out the method of the invention. The volume of the blood sample for carrying out the assay of the invention is advantageously limited, for example of the order of 80 μL, divided into two aliquot portions.

When the blood sample is taken from the subject, it is important to select conditions that can maintain the integrity of the cell population the activation state of which is to be determined, for example by avoiding agitation of the sample and thermal shocks. In particular, the integrity of the platelets must be preserved.

The ELISA (enzyme-linked immunoabsorbent assay) test of the invention employs the operating conditions described in the paragraphs below and in further detail in the examples. It follows the well-known principle of ELISA and can be carried out manually or automatically. It may be a qualitative test for determining the phosphorylation state of a protein or a semi-quantitative test when the platelet reactivity index is determined, or a quantitative test when it includes measurement of a calibrated standard.

In a particular implementation of the invention, the cell population the activity of which is measured is the platelet population.

In a particular implementation of the invention, the intracellular protein the phosphorylation state of which is tested is a protein of the kinase family, or it is a kinase substrate protein.

Thus, the intracellular proteins the phosphorylation state of which is to be determined using the method of the invention are, for example, selected from kinase substrate proteins or kinase itself, selected from the kinases PKA, PKG, AKT, ERK, JAK2, BCR-ABL.

In a preferred implementation of the invention, the intracellular protein the phosphorylation state of which is tested is the protein VASP or STATS.

In another implementation of the invention, the intracellular protein the phosphorylation state of which is to be tested is selected from AKT, p38 and ERK.

The ELISA assay of the invention requires the formation of an immunological complex between the biomarker protein targeted for determining its phosphorylation state and an antibody specifically recognizing this phosphorylated state of said protein. Recognition is termed specific when the antibody under consideration recognizes the protein in question in the phosphorylated state under consideration and does not significantly recognize that same protein in a non-phosphorylated state. The antibody also essentially recognizes the protein in question in the phosphorylated state in question and does not significantly recognize other proteins contained in the treated sample.

The ELISA test of the invention may be carried out in one or two steps. When it is carried out in two steps (using a known standard mode), the treated blood sample is initially brought into contact with an antibody (capture antibody) specifically recognizing the test intracellular protein irrespective of its phosphorylation state. Next, the captured antigen is revealed by bringing it into contact with an antibody (revealing antibody) specifically recognizing the test intracellular protein once it is in the phosphorylated form.

When it is carried out in a single step, the test intracellular protein is brought into contact with the revealing antibody in the well simultaneously with the capture antibody.

Phosphorylation-state dependent antibodies or antibodies that are specific to this phosphorylation state have been obtained for various proteins, for example using the technique described by Czernik A J et al (1991), Methods Enzymol 201: 264. To this end, animals, for example rabbits, may be immunized with phosphorylated proteins or synthetic phosphopeptides representing the amino acid sequence framing the phosphorylation site under consideration in the target protein. Said peptides must be sufficient to constitute an epitope. Said peptides may, for example, contain 6 to 20, 6 to 15, preferably 6 to 10 amino acid residues. Preferably, the antibodies generated are selected from those that have a high affinity and good specificity for the antigen constituted by the phosphorylated protein.

To detect the phosphorylated VASP protein, the preparation of antibody, in particular monoclonal antibody, is described in European patent EP 1 042 368, herein incorporated by reference. In particular in that patent, an effective monoclonal antibody is the antibody produced by the hybridoma 16C2 (clone 16C2) deposited with the DSM Collection under accession number ACC2330. Example 1 of that patent EP 1 042 368 describes the recovery of the phosphorylated anti-VASP antibody (P-VASP). In the context of the present invention, other anti P-VASP antibodies may be generated using the peptide sequence KLRKVS²³⁹KQ or the sequence RKVS²³⁹KQE. Anti P-VASP antibodies are also commercially available. In addition to the antibody described above, mouse monoclonal antibody (clone 4i67) distributed by US Biological or goat polyclonal anti P-VASP antibody, Ser239, Santa Cruz, sc-23507, etc, may be cited.

If necessary, the ELISA is carried out using a second antibody recognizing the first antibody, to improve specificity.

The term “antibody” means an antibody comprising all of the heavy chains and the light chains if appropriate, or a fragment of antibody containing or constituted by the recognition site for the target antigen, for example a fragment constituted by a heavy chain or by an association of heavy chains, for example a (Fab')₂ fragment or a Fab fragment.

In accordance with a particular implementation of the invention, formation of an immunological complex is revealed with a substrate of the antibody marker, for example a colored substrate or a fluorescent marker, and includes a step for measuring the optical density. A marker such as peroxidase may be used.

The optical density is, for example, measured at 450 nm, in particular when the marker for the antibody is a peroxidase.

In particular, the invention can be used to determine the phosphorylation state of an intracellular protein which is a kinase platelet substrate such as the VASP protein phosphorylated at the serine 239 position, or which is a kinase such as the AKT protein phosphorylated at the serine 473 position, STATS phosphorylated at the tyrosine 694 position, p38 phosphorylated at the threonine 180 or tyrosine 182 position, or ERK phosphorylated at the threonine 202 position or at the tyrosine 204 position.

Specific antibodies for the phosphorylated form of these proteins are commercially available or may be prepared by the methods cited in the present application.

As an example, the following antibodies are available:

Anti-p-STATS (Tyr 694) antibody from rabbits, sold with references: 9351L PAb Cell Signaling, clone C71E5 MAb, Cell Signaling, clone C11C5 MAb, Cell Signaling, or with references: clone 47 BD, clone 5G4-MCF-7, Nanotools, clone 14H2, Cell Signaling, clone ST5P-4A9, Zymed, or for the rabbit anti-P-AKT antibody, clone 193H12 sold by Cell Signaling.

Antibodies recognizing the non-phosphorylated form of said proteins, (to constitute the capture antibodies) are also available commercially: for STAT5, a rabbit antibody can be cited (clone C-17, Pab Santa Cruz) or a mouse antibody (clone 89BD, clone ST5-8F7, Invitrogen, clone A-9, Santa Cruz), for AKT a rabbit antibody is distributed by Cell Signaling (clone 193H12), for phosphorylated ERK, specific antibodies are, for example, sold by Cell Signaling (reference clone 197G2), by Calbiochem (reference clone 12D4) or by Lifespan Biosciences (reference LS-C16383). For p38, Cell Signaling also proposes antibodies with references 9228, 9212, 9215, etc), and Santa Cruz proposes antibodies with reference sc-7972 (p38 clone A-12 mouse Mab).

To carry out the ELISA of the invention, the blood sample taken from a patient is treated to prevent it from coagulating. In particular, a normal anti-coagulation agent is added to it to carry out the in vitro assays carried out on blood, for example heparin or sodium citrate, such as trisodium citrate, for example 0.109 M or 0.129 M trisodium citrate in proportions of 9 volumes of blood to 1 volume of anti-coagulation agent.

In particular, the ELISA assay method of the invention comprises carrying out the following steps:

a) activating an aliquot portion of the sample of whole blood with a first compound, advantageously a physiological compound, acting on the cellular activity of a predetermined cell population of said sample by activating the phosphorylation of a predetermined intracellular protein, and activating another aliquot portion with said first compound and with a second compound that inhibits or modifies said phosphorylation, via the pathway of interaction with a cellular receptor or via the pathway of interaction with a cell signaling enzyme;

b) lysis of the cells of the activated sample under conditions that can retain the equilibrium between the non-phosphorylated intracellular protein and said protein in its phosphorylated state, said equilibrium providing evidence of the cellular activity of said predetermined population of cells of the sample;

c) washing to eliminate the lysis products;

d) incubating the treated sample with a capture probe comprising antibodies specifically recognizing the intracellular protein irrespective of its phosphorylation state, said antibody being an immunoglobulin of the IgG type or a F(ab')₂ or Fab fragment;

e) revealing the formation of an immunological complex between the phosphorylated intracellular protein and the revealing antibody specifically recognizing said intracellular protein in its phosphorylated state.

The reagents and the sample are advantageously deposited into the wells of a microplate. The reagents and the sample are deposited into a single well without the need for transfer.

In a particular implementation of the invention, the above steps are modified in that the second compound is a substance that is under investigation as regards inhibition or modification of the phosphorylation state, which is either administered to the patient exclusively before taking the sample, or is brought into contact with the sample of whole blood that has been taken before activation with the first physiological compound described above.

In a particular implementation of the invention, the step for revealing the formation of the immunological complex is followed by a step for determining the platelet reactivity index (PRI) that may be obtained in particular when the immunological complex is detected by reading the optical density associated with revealing the marker for the antibody:

$$PRI = \frac{OD [ C 1 ] - OD [ C 1 + C 2 ]}{OD [ C 1 ] - OD [ blank ]}$$

where C1=first compound, for example phosphorylation activator;

C2=second compound, for example phosphorylation inhibitor.

In a particular implementation of the assay method, the intracellular protein the phosphorylation state of which is to be determined is the VASP protein and the cellular population the activation of which is to be determined is the platelet population, an aliquot portion of the sample being activated with a first VASP phosphorylation agonist, another aliquot portion of the sample being activated with the same first compound and also with a second compound interacting with a target platelet receptor of the test substances for their biological or therapeutic activity.

In another particular implementation of the assay method, the physiological agonist for the phosphorylation of VASP is selected from prostaglandins activating adenylyl cyclase, such as PGE1, PGI2, PDG2 and substances activating guanylyl cyclase, such as NO-donating substances (for example SNP: sodium nitroprusside), or natriuretic peptides (for example ANP: atrial natriuretic peptide; BNP: B-type natriuretic peptide).

In a particular implementation, the compound inhibiting or modifying phosphorylation is a substance interacting with a platelet receptor such as the receptor P2Y12 stimulated by ADP, for example a thienopyridine such as clopidrogel or ticlopidine and/or with a cell signaling enzyme such as adenylyl cyclase, guanylyl cyclase, phosphodiesterases such as PDE2, PDE3 or PDE5, or kinase proteins such as PKA or PKG.

The following examples illustrate the results obtained with substances with activity regarding the phosphorylation of intracellular proteins. These substances are representative of said second compounds, when they are used alone or in association with an additional physiological compound (for example ADP when the signaling pathway observed involves P2Y12).

In a particular implementation of the invention, in addition to the steps defined above, the assay method may include a step for freezing, for example at −20° C. or at −80° C., which is carried out after the washing step, to preserve the sample. The inventors have observed that the sample may be tested after having been preserved in this manner, without altering its stability, and thus means that reliable results can be obtained after preservation by freezing for more than two months following collection.

The inventors have also observed that in the absence of freezing, the samples can be tested for up to 48 h following sampling, since the platelets remain stable.

According to the test, a compound the interaction with platelet activation (test compound) of which is to be observed does not have to be added after taking the blood sample as indicated in the above steps with respect to the “second compound”, but may have been administered to the patient prior to taking the sample. In this scenario, administration of said second compound may either not have been carried out on the sample of whole blood that has been taken if the test compound interacts directly via the pathway involving phosphorylation, or it may consist of administering a physiological compound of the cell signaling pathway regulating phosphorylation, especially when it is necessary to demonstrate the activity of the compound administered to the patient.

When the test compound is a compound which has been administered to the patient, the analysis of the invention may be intended to determine the degree of efficiency of said compound or the patient's resistance to the treatment.

In a preferred implementation of the invention, the assay uses a lysis buffer which comprises or is constituted by the following mixture, or by a functionally equivalent mixture:
2% to 0.02% of the final concentration of sodium dodecyl sulfate (SDS), preferably 0.2%;
5% to 0.2% of the final concentration of Triton X-100 (octylphenoxypolyethoxy phenol), preferably 2%;
1% to 0.004% of the final concentration of a broad spectrum microbicidal agent of the microbicidal type containing, as active principles, 5-chloro-2-methyl-4-isothiazolin-3-one and 2-methyl-4-isothiazolin-3-one, preferably 0.04%.

A functionally equivalent mixture may be prepared by replacing the SDS by another anionic detergent such as cholate or deoxycholate, and/or by replacing the Triton X-100 by another non-ionic detergent such as Nonidet P-40 or Tween 80. The ionic and non-ionic detergents may be used in concentrations of 5% to 0.005%.

An appropriate microbicidal agent for producing the lysis buffer of the invention is ProClin® (Rohm and Haas), ProClin 150 or ProClin 300. It may be replaced by another broad spectrum agent capable of inhibiting the growth of microorganisms and of causing cell death.

Advantageously, when carrying out out the ELISA, all of the incubation steps are carried out at ambient temperature or at a temperature of 37° C. and the lysis step is carried out for a period of 1 to 30 minutes, preferably 10 minutes, at ambient temperature, or at a temperature of 37° C.

Other characteristics and advantages of the invention will become apparent from the figures and the examples.

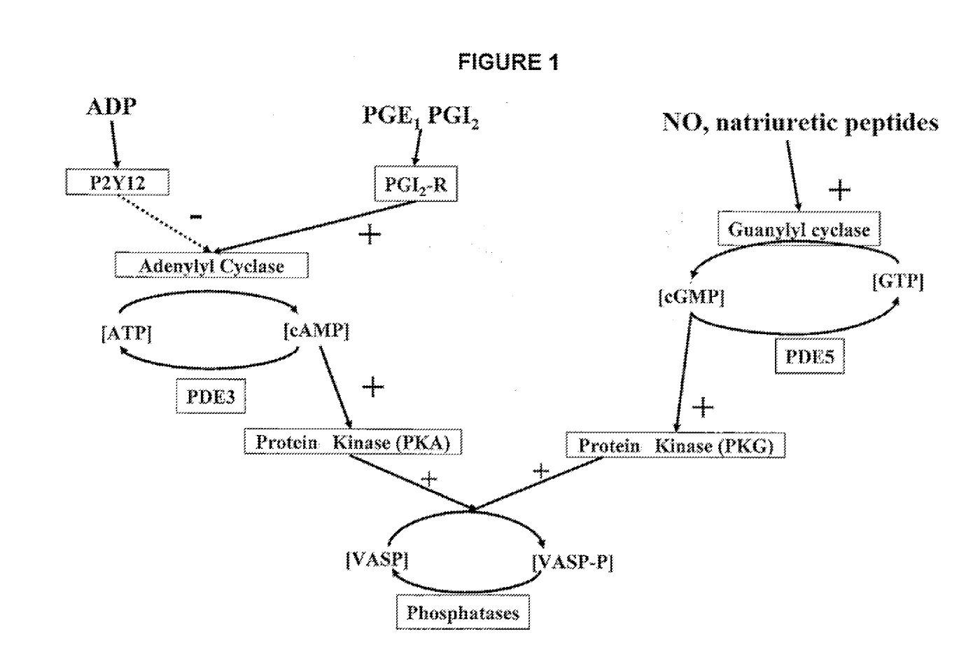

FIG. 1: simplified diagram of the VASP phosphorylation regulation pathway (cascade);

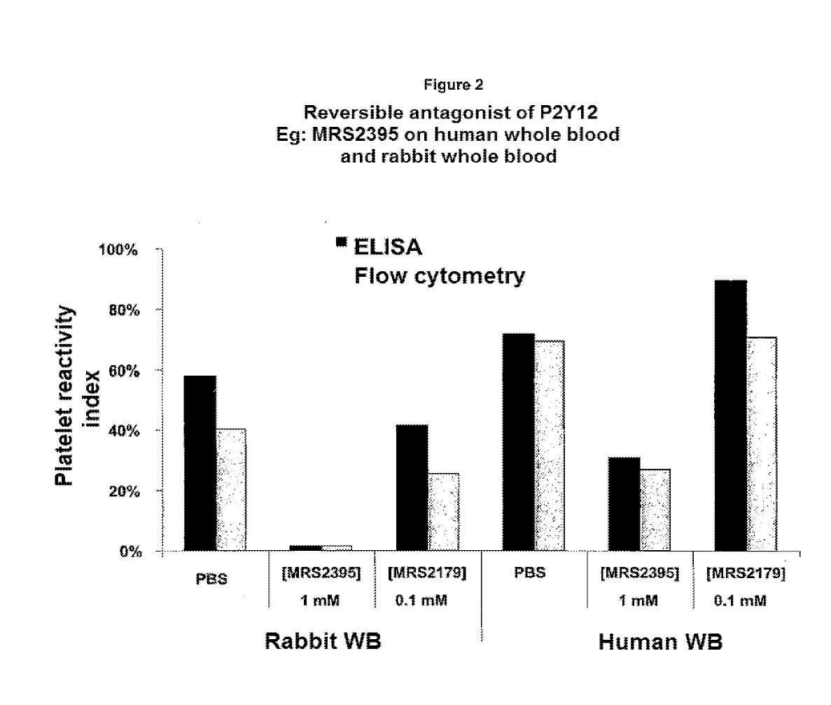

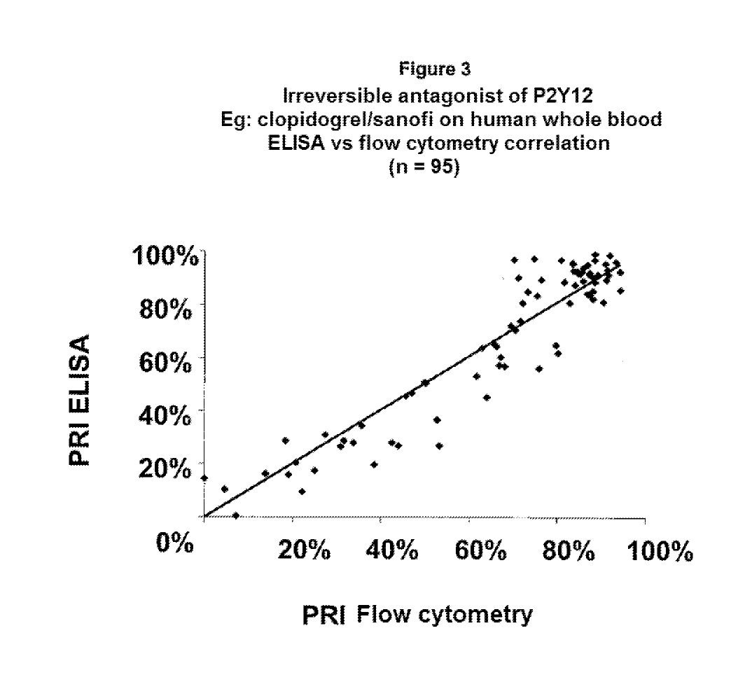

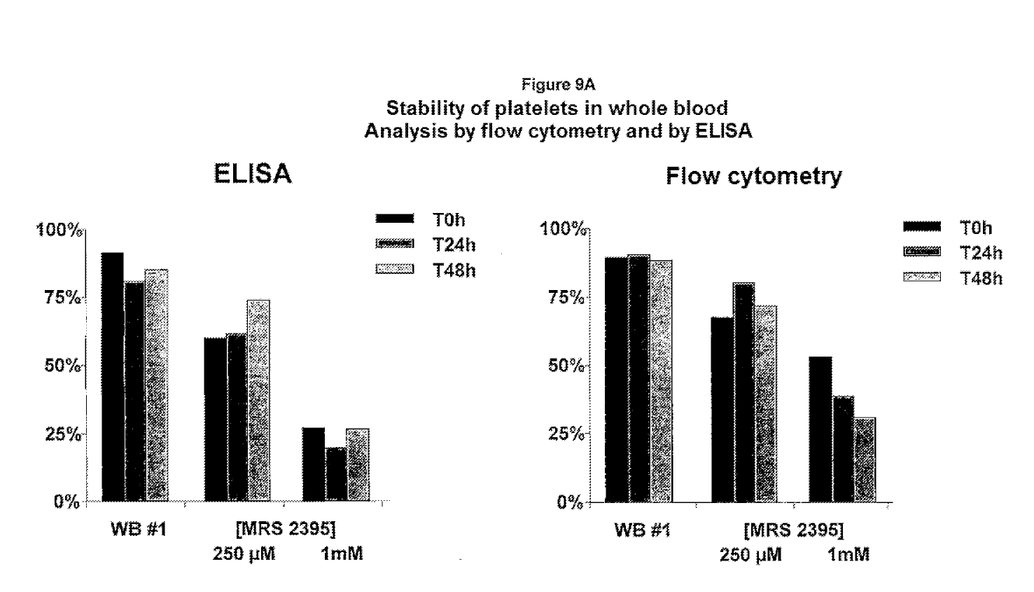

FIGS. 2 to 8: effect of various active substances on platelet activation via an interaction with the intracellular protein phosphorylation pathway;

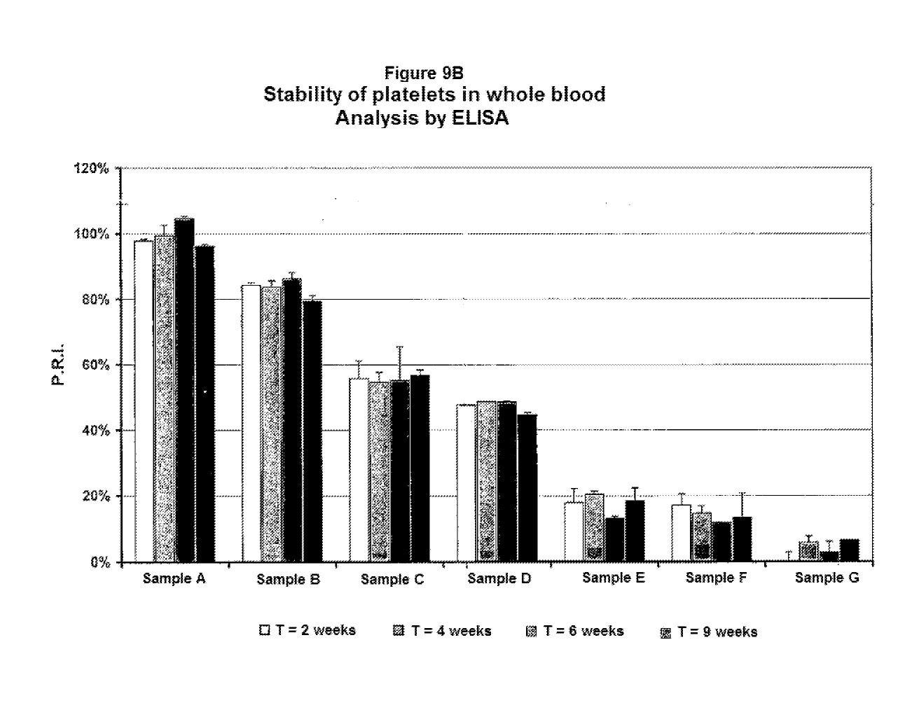

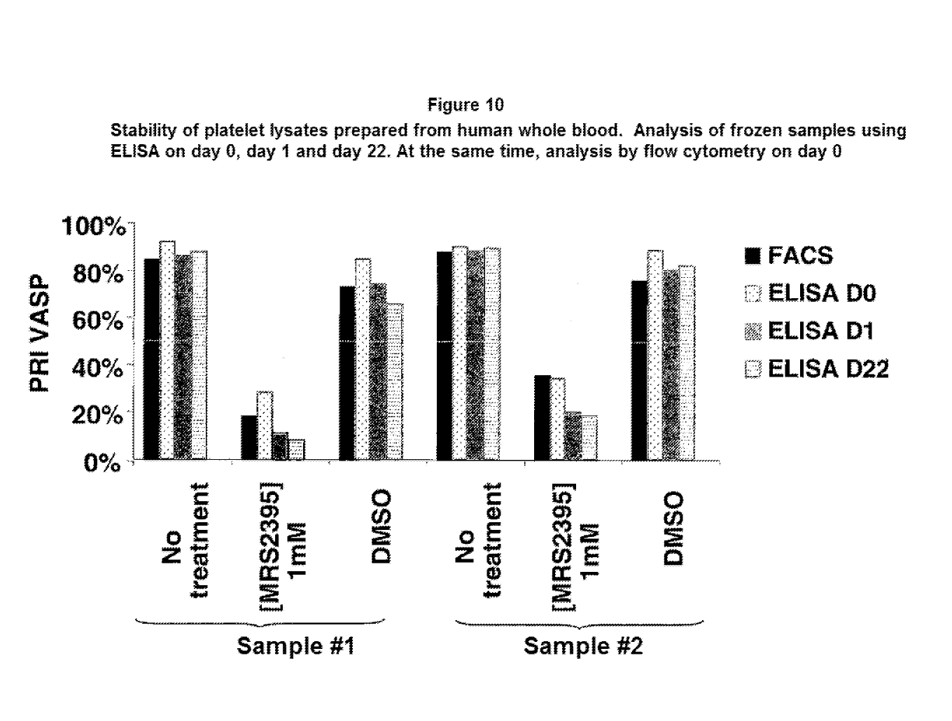

FIGS. 9, 10: study of platelet stability before treatment or after treatment, lysis and freezing;

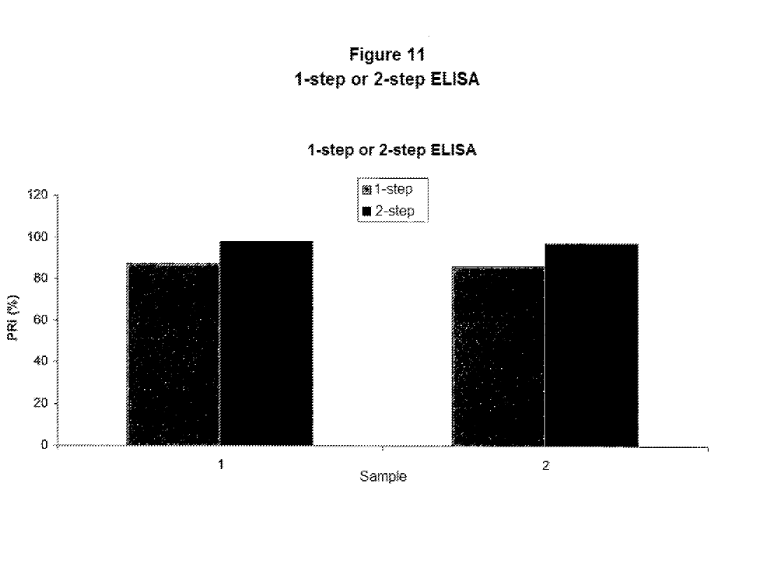

FIG. 11: results when ELISA is carried out in one or two steps.

### EXAMPLES

I. Analysis of platelet activation by phosphorylation of the VASP protein in a whole blood ELISA test to demonstrate the influence of activation of the P2Y12 receptor on the phosphorylation state of VASP

Even though it is well established that in platelets, the phosphorylation of VASP (P-VASP) may mirror the activation state of the functional ADP receptor (P2Y12), the consequences of lysis of all of the blood cells (red globules+leukocytes) contained in whole blood on platelet activation mediated by P2Y12 has not been documented until now.

In particular, release into the medium of enzymes involved in all of the levels of signaling included between the receptor (for example: P2Y12) and a substrate that is susceptible of being phosphorylated constituting a biomarker (for example; VASP) may potentially perturb the biomarker [symbol] phosphorylated biomarker equilibrium (for example: VASP [symbol] P-VASP or any other signaling biomarker such as AKT, STATS, p38, ERK, etc). Among the ubiquitous enzymes released into the medium by the action of cell lysis, the protein kinases (PKA, PKG) and phosphatases are responsible for the phosphorylation of VASP or for its dephosphorylation. Thus, it can be directly expected that the action of these enzymes may modify the equilibrium (P-VASP [symbol] VASP) directly during the cell lysis step, thereby altering the relevance of the results of the measurement.

Thus, the inventors designed the ELISA analysis so as to:

1/ fix the activity of the cellular enzymes released into the medium, along with cell lysis;

2/ render ineffective the effect of the nucleotides (ADP, ATP, AMP, cAMP, GMP, cGMP, etc) liberated by the action of cell lysis. As an example, it is well known that red globules are ADP reservoirs, which may have an immediate impact on cell activation, and in particular on the platelets.

The inventors have observed that it is possible to overcome these risks of interference in the analysis, as illustrated by the ELISA analysis designed in the context of the present application and in particular in the following example.

Microwell plate operating protocol
For ELISA analysis of the phosphorylation state of VASP under the control of the activator PGE1 and the receptor P2Y12 activated by ADP, the following steps and conditions were employed:
1 Blood was taken into sample tubes containing an anti-coagulation agent such as Na citrate (0.109 M or 0.129 M);
2. a) if necessary, a compound for which the interaction with the platelet activity is to be tested was deposited in a portion of the wells containing the whole blood sample;
2. b) the PGE1 or PGE1 +ADP activator (40 μL) was deposited in the corresponding wells of the microplates;
3 The blood (40 μL) was added, then it was agitated by means of a series of intakes and discharges;
4 The mixture was incubated for 10 min at ambient temperature;
5 The lysis buffer (100 μL) was added to the wells containing the activated blood and it was all agitated by means of a series of intakes and discharges. Lysis buffer (180 μL) was also deposited in the “blank” wells;
6 The reaction medium was incubated for 30 minutes at ambient temperature;
7 The plate was then washed three times with diluted washing solution (3×300 μL, supplied 20X concentrated);
8 The peroxidase probe comprising the 20X concentrated anti-P-VASP antibody was diluted in dilution buffer;
9 The diluted peroxidase probe was deposited into all of the wells (200 μL);
10 It was incubated for 30 minutes at ambient temperature;
11 the plate was washed three times with diluted washing solution (3×300 μL, supplied 20X concentrated);
12 TMB revealing substrate was then deposited into all of the wells (200 μL);
13 It was incubated for 5 min at ambient temperature;
14 Next, stop solution was added to all of the wells (100 μL);
15 and the plate was left to stand for several minutes;
16 The OD₄₂₀ₙₘ was measured using a plate reader;
17 The Platelet Reactivity Index was calculated using the following formula:

$$PRI = \frac{{OD}_{450 nm} [ PGE 1 ] - {OD}_{450 nm} [ PGE 1 + ADP ]}{{OD}_{450 nm} [ PGE 1 ] - {OD}_{450 nm} [ blank ]}$$

The composition of the reagents used in the protocol was as follows:

1. ELISA plate:

NUNC “Maxisorp” plate coated with a total anti-VASP F(ab')₂ (clone 1E273) from immunoGlobe, Germany (http://www.immunoglobe.com).

2. Lyophilized PGE1 activator:

Prostaglandin E1 (5 μM) from Carbomer (http://www.carbomer.com)+excipients.

3. Lyophilized PGE1+ADP activator:

Prostaglandin E1 (5 μM) from Carbomer (http://www.carbomer.com). The molarity could be modified within the range 5 pM-5 mM;

+ADP (20 μM) from Sigma (http://www.sigmaaldrich.com). The molarity could be modified within the range 5 pM-5 mM.

+excipients.

4. Lysis buffer

PBS buffer containing.

2% of Triton X-100 (Sigma, http sigmaaldrich.com). The concentration could be modified within the range 0.002%-10%.

0.2% of SDS (Sigma, http://www.sigmaaldrich.com). The concentration could be modified within the range 0.0002%-10%.

0.4% of ProClin 300 (Sigma, http://www.sigmaaldrich.com). The concentration could be modified within the range 0.000002%-10%.

5. 20X washing solution

| Compound | Concentration, g/l |
|---|---|
| Sodium di hydrogen phosphate, 2 H₂O | 31.13 |
| Sodium chloride | 174.90 |
| Tween 20 | 19.95 |
| Nipagine A | 0.998 |
| Gentamycin sulfate | 0.075 |
| Light green SF yellowish | 0.695 |
| Hydrochloric acid | Not quantifiable (for |
| Sodium hydroxide | adjustment of pH) |

Final pH = 6.65 ± 0.05
Final density: 1.14

6. Dilution buffer

Ready-to-use dilution buffer, quantities for 1 liter:

| | | |
|---|---|---|
| NaCl | 8.8 | g |
| NaH₂PO₄ | 3.9 | g |
| Tween 20 | 1 | g |
| Bovine albumin | 1 | g |
| Acid blue 25 | 12.5 | mg |
| proClin | 0.5 | g |

pH: target 7.50 (7.30-7.70)

7. Peroxidase 20X probe

This was a mouse monoclonal phosphorylated anti-VASP antibody (clone 16C2) described in patent EP 1 042 368 and available from NanoTools, Germany (http://www.nanotools.de) coupled with a HRP peroxidase enzyme: peroxidase (POD) from Roche, Switzerland (http://www.roche-applied-science.com) or BBI Enzymes, formerly Biozyme http://www.biozyme.com).

### 8. TMB

“TMB ONE, ready-to-use substrate” from Kem-En-Tec, Denmark, (http://www.kem-en-tec.com).

9. Stop solution

### H₂SO₄=0.2 M

The molarity could vary in the range of 0.0002 M-10 M.

The analysis of the sample that was taken could at the same time include that of a normal sample.

II. Analysis of platelet activation by phosphorylation of the VASP protein in a whole blood ELISA test to evaluate the influence of substance acting on the receptors and/or enzymes of the VASP phosphorylation pathway
II.1 PDE3 inhibitor test

Cilostazol (Angilillo, USA), which is an active principle that is active on phosphodiesterase PDE3, and used to monitor platelet activation linked to pathologies such as diabetes, was tested on whole human blood.

100 μL of whole blood and 100 μ/L of cilostazol dilution were used. Incubation was carried out for 2h30 at 37° C.

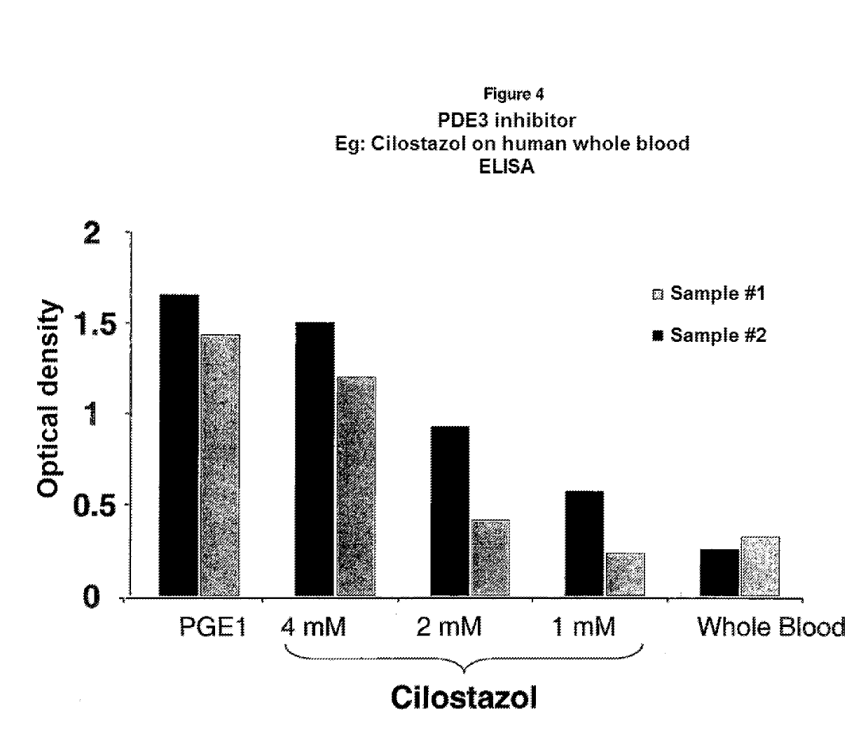

The results shown in FIG. 4 show the change in the phosphorylation state of VASP in the presence of different quantities of cilostazol.

Other molecules can be tested in similar manner.

11.2 EP3 receptor of PGE2 activator test

The active principle Sulprostone, acting on the EP3 receptor of prostaglandin, PGE2, was tested using ELISA on whole blood.

10 μL (FACS) or 40 μL (ELISA) whole blood was used and 10 μL (FACS) or 40 μL (ELISA) of Sulprostone dilution. Incubation was carried out for 10 minutes at ambient temperature.

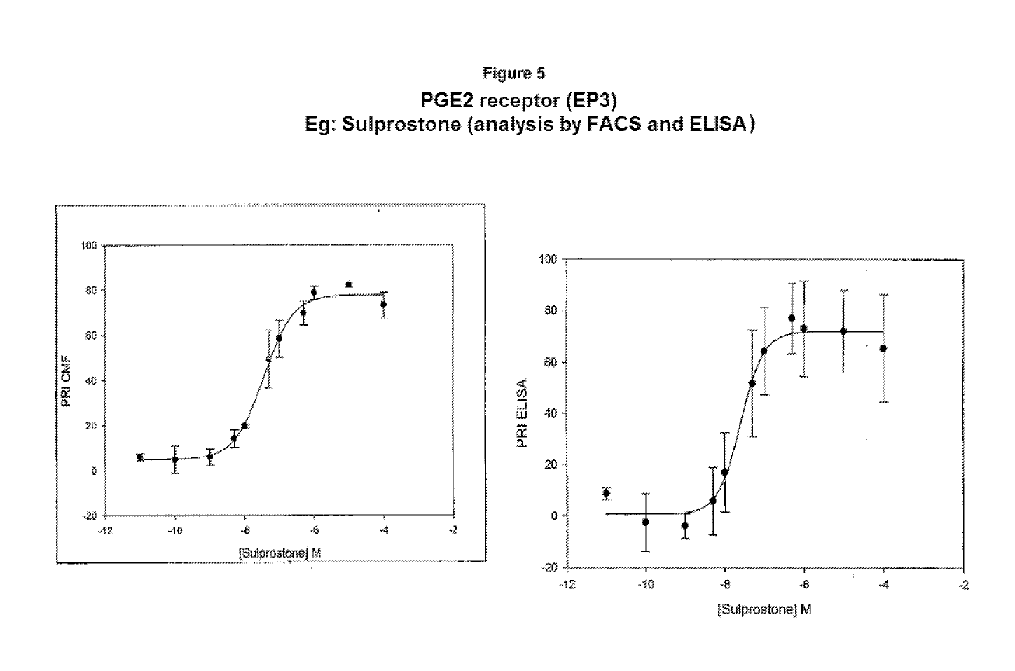

Its effect on the VASP phosphorylation is shown in FIG. 5.

11.3 PGD2 receptor activator test

A compound designated BW245c, acting on the DP1 receptor of PGD2, was analyzed using FACS and ELISA on whole blood.

10 μL (FACS) or 40 μL (ELISA) of whole blood and 10 μL (FACS) or 40 μL (ELISA) of BW245c dilution were used. Incubation was carried out for 10 minutes at ambient temperature.

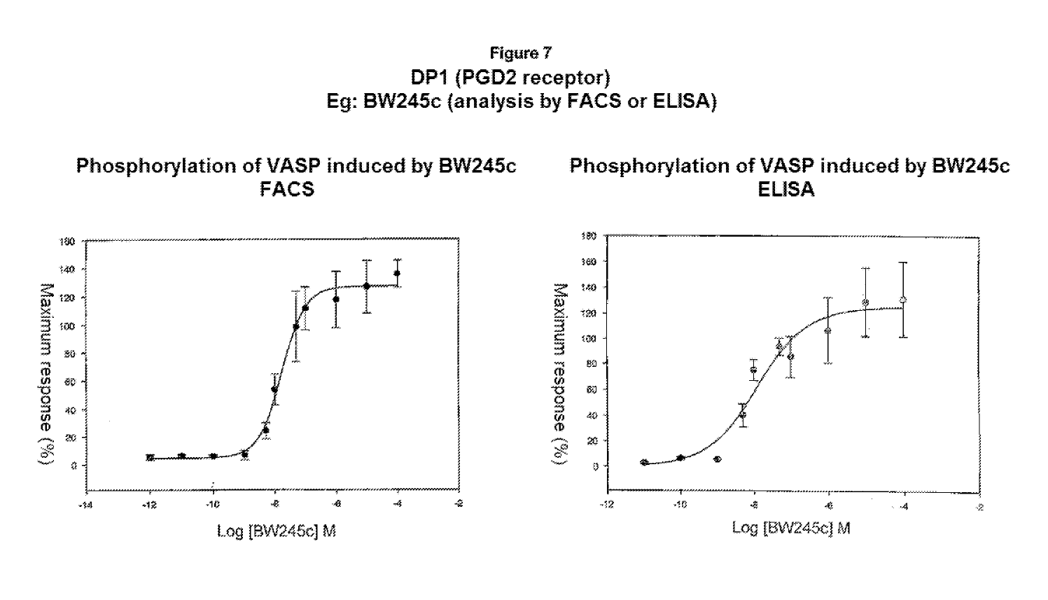

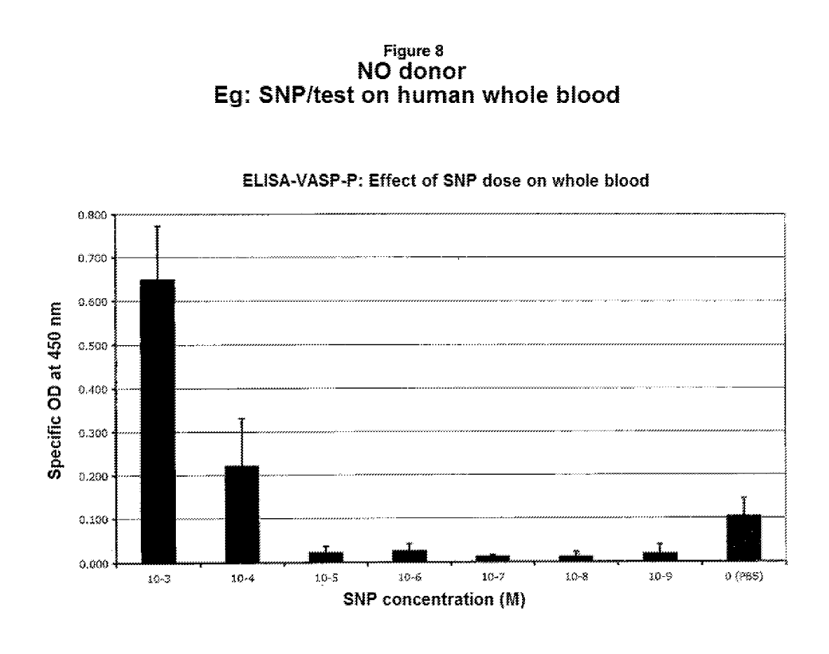

The results are reported in FIG. 7.

III. Analysis of platelet activation by phosphorylation of the VASP protein in a whole blood ELISA test to evaluate the influence of substance acting on the receptors and/or enzymes of the VASP phosphorylation pathway
1 Blood was taken into sample tubes containing an anti-coagulation agent such as Na citrate (0.109 M or 0.129 M);

2 a) if necessary, a compound the interaction with the platelet activity of which was to be tested was deposited in a portion of the wells containing the whole blood sample;

2 b) the PGE1 or PGE1+TRAP14 activator (40 μL) was deposited in the corresponding wells of the microplates;

3 The blood (40 μL) was added then agitated using a series of intakes and discharges;

4 The mixture was incubated for 10 min at ambient temperature;

5 The lysis buffer (100 μL) was added to the wells containing the activated blood and it was all agitated by means of a series of intakes and discharges. Lysis buffer (180 μL) was also deposited in the “blank” wells;

6 The reaction medium was incubated for 30 minutes at ambient temperature;

7 The plate was then washed three times with diluted washing solution (3×300 μL, supplied 20X concentrated);

The composition of the reagents is given in point II.

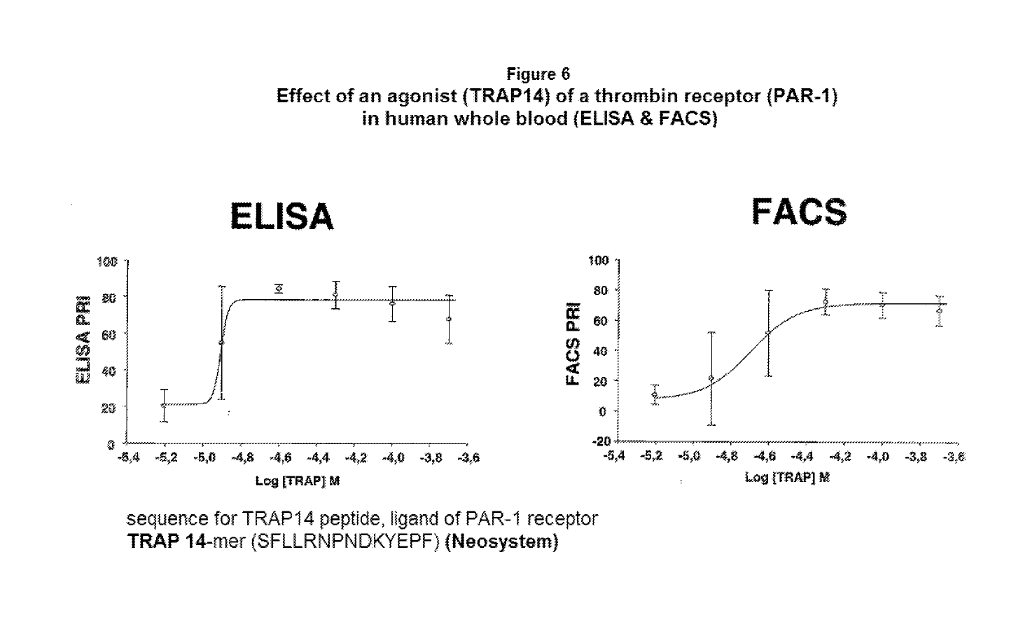

The TRAP14 agonist of the thrombin receptor (PAR-1) was tested on the one hand using ELISA, and on the other hand using FACS, on whole blood, to evaluate its indirect effect on the phosphorylation state of the VASP protein. The results are shown in FIG. 6.

The compound C1 (first compound) was PGE1 and the compound C2 (second compound) was TRAP14.

IV. Preservation of whole blood samples after activation (by PGE1+ADP+MRS2395)

FIGS. 9 and 10 show the stability of platelets for whole blood samples (WB) treated with PGE1+ADP+MRS2395, up to 48 h after treatment or, after lysis then freezing, for up to 22 days. The results of the PRI obtained using ELISA and flow cytometry are shown.

1. A method for assay by ELISA, carried out on a whole blood sample, of the cellular activation state of a predetermined population of cells of the sample, comprising determining the phosphorylation state of an intracellular protein involved in a cell signaling pathway (biomarker).

2. A method for assay by ELISA according to claim 1, in which the cellular activation state that is assayed is that of the platelets.

3. A method for assay by ELISA according to claim 1, in which the intracellular protein the phosphorylation state of which is tested is a protein of the kinase family, or it is a kinase substrate protein.

4. A method for assay by ELISA according to claim 2, in which the intracellular protein is a substrate protein of a kinase or the kinase itself, selected from the kinases PKA, PKG, AKT, ERK, JAK2, BCR-ABL.

5. A method for assay by ELISA according to claim 1, in which the intracellular protein is a platelet kinase substrate such as the protein VASP or STATS, or it is a kinase such as the protein AKT, p38 or ERK.

6. A method for assay by ELISA according to claim 1, comprising the following steps carried out on a sample of whole blood:
a) activating an aliquot portion of the sample of total blood with a first compound, advantageously a physiological compound, acting on the cellular activity of a predetermined cell population of said sample by activating the phosphorylation of a predetermined intracellular protein, and activating another aliquot portion with said first compound and with a second compound that inhibits or modifies said phosphorylation, via the pathway of interaction with a cellular receptor or via the pathway of interaction with a cell signaling enzyme;
b) lysis of the cells of the activated sample under conditions that can retain the equilibrium between the non-phosphorylated intracellular protein and said protein in its phosphorylated state, said equilibrium providing evidence of the cellular activity of said predetermined population of cells of the sample;
c) washing to eliminate the lysis products;
d) incubating the treated sample with a capture probe comprising antibodies specifically recognizing the intracellular protein irrespective of its phosphorylation state;
e) revealing the formation of an immunological complex between the phosphorylated intracellular protein and the revealing antibody specifically recognizing said intracellular protein in its phosphorylated state;
said antibody being labeled if appropriate.

7. A method for assay by ELISA according to claim 1, comprising the following steps carried out on a whole blood sample taken from a patient to whom a compound interacting with platelet activation by modification to the phosphorylation of a predetermined intracellular protein (termed the second compound) has been administered;
a) activating an aliquot portion of the sample of total blood with a first compound acting on the cellular activity of a predetermined cell population of said sample by activating the phosphorylation of a predetermined intracellular protein;
b) lysis of the cells of the activated sample under conditions that can retain the equilibrium between the non-phosphorylated intracellular protein and said protein in its phosphorylated state, said equilibrium providing evidence of the cellular activity of said predetermined population of cells of the sample;
c) washing to eliminate the lysis products;
d) incubating the treated sample with a capture probe comprising antibodies specifically recognizing the intracellular protein irrespective of its phosphorylation state;
e) revealing the formation of an immunological complex between the phosphorylated intracellular protein and the revealing antibody specifically recognizing said intracellular protein in its phosphorylated state;
said antibody being labeled if appropriate.

8. A method according to claim 6, in which activation with said second compound precedes activation with said first compound.

9. An assay method according to claim 6 characterized in that, in step a), activation with said second compound inhibiting or modifying the phosphorylation is a substance for which the effect on cellular activity is to be tested, for example a substance with a biological or therapeutic activity.

10. A method for assay by ELISA according to claim 6, in which the washing step is followed by a freezing step to preserve the sample.

11. A method for assay by ELISA according to claim 6, in which revealing the formation of the immunological complex is carried out with a substrate for the marker of the antibody and comprises a step for measuring the optical density of the two aliquot portions of the sample that are treated, followed if appropriate by determining the PRI.

12. An assay method according to claim 1, in which the intracellular protein the phosphorylation state of which is determined is the VASP protein and the cellular population the activation of which is determined is the platelet population, an aliquot portion of the sample being activated with a first compound that is an agonist of VASP phosphorylation, another aliquot portion of the sample being activated with the same first compound and further with a second compound interacting with a target platelet receptor for substances tested for their biological or therapeutic activity.

13. An assay method according to claim 12, in which the physiological agonist of VASP phosphorylation is selected from enzymes activating adenylyl cyclase such as the prostaglandins PGE1, PGI2 and substances activating guanylyl cyclase such as NO-donating substances or natriuretic peptides.

14. An assay method according to claim 13, in which the compound inhibiting or modifying phosphorylation is a substance interacting with a platelet receptor such as the receptor P2Y12 stimulated by ADP and/or with a cell signaling enzyme such as adenylyl cyclase, guanylyl cyclase, phosphodiesterases such as PDE2, PDE3 or PDE5, or kinase proteins such as PKA or PKG.

15. An assay method according to claim 1, in which the intracellular protein the phosphorylation state of which is determined is the VASP protein and the cellular population the activity of which is determined is the platelet population, an aliquot portion of the sample being activated with a first physiological agonist of VASP phosphorylation, such as PGE1, PDG2 or PGI2, and another aliquot portion being activated with the same first agonist and also with a second compound interacting with a cell receptor, for example interacting with the receptor P2 stimulated by the nucleotide ADP, said second compound inhibiting VASP phosphorylation or being tested to investigate its inhibition properties.

16. An assay method according to claim 1, in which the antibodies specifically recognizing the intracellular protein in its phosphorylated state are monoclonal antibodies, for example monoclonal antibodies specifically recognizing the VASP protein phosphorylated at serine 239, or monoclonal antibodies specifically recognizing the AKT protein phosphorylated at serine 473.

17. An assay method according to claim 1, in which the lysis buffer comprises or is constituted by the following mixture:
0.0002% to 10%, preferably 2% to 0.02% of the final concentration of sodium dodecyl sulfate (SDS), preferably 0.2%;
0.002% to 10%, preferably 5% to 0.2% of the final concentration of Triton X-100 (octylphenoxypolyethoxyphenol), preferably 2%;
0.000002% to 10%, preferably 0.004% to 1% of the final concentration of a broad spectrum microbicidal agent of the microbicidal type containing, as active principles, 5-chloro-2-methyl-4-isothiazolin-3-one and 2-methyl-4-isothiazolin-3-one, preferably 0.04%.

18. An assay method according to claim 1, in which all of the incubation steps are carried out at ambient temperature or at a temperature of approximately 37° C. and the lysis step is carried out for a period of 10 to 30 minutes, preferably 10 minutes, at ambient temperature, or at a temperature of approximately 37° C.

19. An assay method according to claim 1, in which the blood sample has been collected under conditions preserving the integrity of the cellular population the activity of which is to be tested, in particular under conditions preventing activation of said cellular population during collection, and the collected sample is then treated with an anti-coagulation agent such as sodium citrate, preferably 0.109 M or 0.129 M trisodium citrate, in proportions of 9 volumes of blood to 1 volume of anti-coagulation agent.

20. An assay method according to claim 1, applied to a whole blood sample that has already been collected from a patient treated with an agonist agent or with an antagonist agent for the activity of a predetermined population of cells contained in the sample.

21. An assay method according to claim 19, in which the agonist agent or the antagonist agent acts on platelet aggregation.

22. An assay method according to claim 20, in which the antagonist of platelet activation is an antagonist of the P2Y12 receptor to ADP, for example a thienopyridine such as clopidrogrel or ticlopidine, or it is a substance interacting with a cell signaling enzyme such as adenylyl cyclase, guanylyl cyclase, phosphodiesterases such as PDE2, PDE3 or PDE5, or kinase proteins such as PKA or PKG.

23. An in vitro method for monitoring coagulation problems in a patient treated with an antagonist agent of platelet activation, characterized in that it comprises assaying in accordance with the method according to claim 1, determining the platelet reactivity index (PRI) and interpreting the PRI, for example in order to determine whether the patient responds well or poorly to said antagonist agent.

24. A kit for carrying out a method for assaying by ELISA, carried out on a whole blood sample, of the cellular activation state of a predetermined population of cells of the sample, characterized in that it comprises:
a lysis buffer as defined in claim 17;
a washing solution;
an antibody specifically recognizing an intracellular protein in its phosphorylated state, labeled if necessary.
